(19) United States
(12) Patent Application Publication
(10) Pub. No.: US 2009/0130934 A1
(43) Pub. Date: May 21, 2009

(54) Esterquats Containing OH Groups For Improving Fragrance Effect

(75) Inventors: Andreas Schmidt, Langenfeld (DE); Christian Mueller, Osterspai (DE); Konstanze Mayer, Duesseldorf (DE); Rainer Jeschke, Duesseldorf (DE)

Correspondence Address: Ratner Prestia, P.O. Box 980, Valley Forge, PA, 19482, US

(73) Assignee: Henkel AG & Co. KGaA, Duesseldorf (DE)

(21) Appl. No.: 12/356,951

(22) Filed: Jan. 21, 2009

Related U.S. Application Data
(63) Continuation of application No. PCT/EP2007/055174, filed on May 29, 2007.

(30) Foreign Application Priority Data
Jul. 25, 2006 (DE) 10 2006 034 899.0

(51) Int. Cl. C11D 3/50; A61K 8/31; A61K 8/34; A61K 8/35; A61K 8/36; A61K 8/37; A61Q 13/00; B32B 5/02

(52) U.S. Cl. 442/96; 510/101; 510/107; 512/26; 512/25; 512/27; 512/20; 512/1; 512/4

(57) ABSTRACT

Agents for treating textiles or surfaces, which contain at least one fragrance and at least one esterquat with the general formula (I): [N⁺R₁R₂R₃R₄]X⁻ that contains an OH group. The use of agents for treating textiles or surfaces to prolong the fragrant scent of detergents, cleaning agents, fabric softeners, or solid surfaces treated with these agents, and method for producing them.

### CROSS-REFERENCE TO RELATED APPLICATIONS

This application is a continuation under 35 U.S.C. §§ 120 and 365(c) of International Application PCT/EP2007/055174, filed on May 29, 2007. This application also claims priority under 35 U.S.C. § 119 of DE 10 2006 034 899.0 filed on Jul. 25, 2006. The disclosures of PCT/EP2007/055174 and DE 10 2006 034 889.0 are incorporated by reference in their entirety.

### BACKGROUND OF THE INVENTION

Agents for treating textiles and surfaces usually comprise fragrances that lend the agent a pleasant and fresh odor. The fragrances, both synthetic as well as natural, usually mask the inherent fragrances of the other ingredients in the composition. Consequently, cosmetics, soaps, sanitary articles, agents for treating surfaces and other products are perfumed in order that they elicit a pleasant and clean sensation of odor with the consumer. In the laundry detergent field, fragrances are particularly important ingredients of the composition. The washing should have a pleasant and fresh fragrance both when wet and when dry. Consequently the fragrances are required to possess a good absorbance capability onto the fibers and remain bound thereto so as to be subsequently released again after a delay, such that the washing emits a pleasant fragrance over a longer period of time. Therefore, the demands on the fragrances are very stringent. The fundamental problem with the use of fragrances is that fragrances are highly volatile substances. However, this property is also responsible for its fragrant effect. Consequently, with the use of fragrances in agents for treating textiles and surfaces, one is faced with the challenge of adequately stabilizing these highly volatile fragrances such that they do not all evaporate extremely rapidly leaving no more fragrant effect. The fragrances should evaporate in a controlled manner and thereby effect a long lasting and as constant as possible fragrant effect. A further problem is the fact that the fragrant impression of a perfume changes over time because the odoriferous substances that represent the fresh and high notes of the perfume due to their high vapor pressures evaporate more quickly than the fragrances that represent the heart and body notes.

There already exist several proposals in the prior art to modify fragrances in such a way that they are released over a longer period of time in a controlled manner. For example, in order to achieve a controlled release of fragrances, fragrances are deposited on carrier materials, encapsulated or embedded in compounds. A further variant consists in chemically bonding fragrances to other molecules. The bond between fragrance and molecule is subsequently cleaved, for example hydrolytically, whereby the fragrance is then slowly released again. An example of such a chemical modification of fragrance molecules is the esterification of fragrance alcohols. Such compounds are often called in the prior art pro-fragrances, pro-accords and the like. There exists a broad prior art for the various proposals just cited. Thus, pro-fragrances that comprise a fragrant aldehyde or a fragrant ketone bonded in the form of an oxazolidine are described in U.S. Pat. No. 6,861,402. Thus for example, N-benzene ethanolamine is treated with a fragrance affording a monocyclic oxazolidine. DE-A-1 133 847 for example relates to the use of the condensation products of aldehydes and ketones with oxyamines in perfumery. For this the aldehydes and ketones are treated with ethanolamine or diethanolamine.

In the above-mentioned patents, attempts were made to solve the controlled release of fragrances over an extended period of time by means of the concept of a precursor in the form of pro-fragrances or pro-accords. However, the pro-fragrances are very laborious to synthesize and it is still difficult to control the cleavage kinetics of the substances described therein, with the result that the fragrances are still released too early.

### DESCRIPTION OF THE INVENTION

The invention relates to agents for treating textiles or surfaces, which contain at least one fragrance and at least one esterquat that contains an OH group. The invention also relates to the use of agents for treating textiles or surfaces, and processes for their manufacture.

Accordingly, an object of the present invention is to provide agents for treating textiles or surfaces, which elicit an improved longer fragrance perception by the consumer by means of delayed release (cleavage) of fragrances.

Another object of the present invention was to stabilize fragrances in such a way that they are better absorbed onto and remain attached to the fibers, thereby enabling the fragrances to be released over a longer period of time.

This object is achieved by agents for treating textiles or surfaces, which comprise at least one fragrance and at least one esterquat that contains an OH group. Esterquats have been known for a long time in the prior art. The term esterquat stands for a collective name for cationic surface active compounds containing two hydrophobic groups which are linked through ester bonds with a quaternized di(tri)ethanolamine or with an analogous compound.

Esterquats find use as fabric softeners and have replaced distearyldimethylammonium chloride that was previously dominant in this field because of its unsatisfactory biodegradability.

It has now been surprisingly shown that OH group-containing esterquats according to Formula (I):

[N⁺R₁R₂R₃R₄]X⁻ (I)

introduced into agents for treating textiles or surfaces increase the fragrance yield e.g. on textiles and significantly improve the fragrance delay.

Accordingly, the inventive agents for treating textiles or surfaces comprise at least one fragrance and at least one OH group-containing esterquat of the general Formula (I):

[N⁺R₁R₂R₃R₄]X⁻ (I)

in which
R₁ stands for an alkyl group containing 1 to 4 carbon atoms or hydroxyalkyl group containing 1 to 4 carbon atoms,
R₂, R₃, R₄ independently of one another stand for an alkyl group containing 1 to 4 carbon atoms, hydroxyalkyl group containing 1 to 4 carbon atoms,

—(CH₂)ₘ-A-C(O)-Z or —(CH₂)ₘ—C(O)-A-Z,

wherein A stands for —O, —S—, —NR₅—
with R₅═H or an alkyl group containing 1 to 4 carbon atoms,
Z for a saturated or unsaturated alkyl group containing 8 to 22 carbon atoms, which comprises at least one OH group in the side chain,
m for a whole number in the range 1 to 3 and
X for an anion of an inorganic or organic acid,
with the proviso that at least one group R₂, R₃ or R₄ stands for

—(CH₂)ₘ-A-C(O)-Z or —(CH₂)ₘ—C(O)-A-Z.

Preferred hydroxyalkyl groups are here the hydroxyalkyls with methyl, ethyl, propyl and butyl, quite particularly preferably hydroxyethyl or hydroxypropyl. Further preferred compounds are the compounds in which R₂, R₃ and R₄, quite particularly preferably if R₂ and R₃ stand for —(CH₂)ₘ-A-C(O)-Z or if only R₂ stands for —(CH₂)ₘ-A-C(O)-Z, wherein A is preferably —O.

Further preferred are compounds, in which the alkyl group Z that comprises at least one OH group in the side chain, is selected from the group consisting of unbranched saturated or unsaturated C₈—, C₉—, C₁₀—, C₁₁—, C₁₂—, C₁₃—, C₁₄—, C₁₅—, C₁₆—, C₁₇—, C₁₈—, C₁₉—, C₂₀—, C₂₁—, C₂₂— groups, particularly preferably unbranched saturated or unsaturated C₁₄—, C₁₅—, C₁₆—, C₁₇—, C₁₈—, C₁₉—, C₂₀—, C₂₁—, C₂₂— groups, quite particularly preferably unbranched saturated or unsaturated C₁₆—, C₁₇—, C₁₈— groups. The alkyl group preferably possesses 0, 1, 2, 3 or 4 double bonds. The alkyl group Z quite particularly preferably possesses 0, 1 or 2 double bonds.

In all cases, the alkyl group Z possesses at least one OH group in any position. OH groups on an unbranched saturated or unsaturated C₁₇ alkyl chain are particularly preferred.

OH groups on the C₁₁ atom of an unbranched saturated or unsaturated C₁₇ alkyl chain are preferred. In the case of the unsaturated C₁₇ alkyl chain, it is quite particularly preferred if the double bond in this case is at the C-8 atom. Further preferred are OH groups on an unbranched polyunsaturated C₁₇ alkyl chain, wherein the double bonds are particularly preferably on the C-18 and C-11 atoms. Further preferred are OH groups on an unbranched polyunsaturated C₁₇-alkyl chain, wherein the double bonds are particularly preferably on the C-8 and C-11 and C-14 atoms. Further preferred are OH groups on an unbranched unsaturated C₂₁ alkyl chain, wherein the double bond is particularly preferably on the C-12 atom.

The degree of OH substitution of the at least one OH group-containing esterquats is therefore preferably 1 to 3, quite particularly preferably 1.7 to 2.2. The degree of OH substitution reflects the number of OH groups per side chain in the OH group-containing esterquats. An ester side chain preferably possesses 1 to 2 OH groups. Therefore, an esterquat molecule of this type preferably possesses six OH groups, particularly preferably 2 OH groups. Moreover, an esterquat molecule of this type can also possess a different number of OH groups on different substituents Z. This occurs particularly when R₂, R₃ and/or R₄ stand for —(CH₂)ₘ-A-C(O)-Z for example. Accordingly, the preferred degree of OH substitution is here 1.7 to 2.2. The degree of OH substitution in this case reflects the average number of OH groups that are present per side chain in the molecule.

Accordingly, the inventive agents for treating textiles or surfaces comprise at least one esterquat that carries an OH group in at least one side chain of the ester. Thus the total quantity of the at least one OH group-containing esterquat is between 0.5 to 40 wt. %, preferably between 2.5 and 30 wt. %, particularly preferably between 3.5 and 20 wt. % based on the total quantity of the composition.

The anion X in Formula (I) is preferably an inorganic or organic acid, which can be a halide, sulfate, methosulfate, phosphate, formate, propionate or acetate. The chloride ion is particularly preferred as the halide.

Preferably employed fragrances or perfume oils that can be incorporated into the compositions are not subject to any limitations. Individual odoriferous compounds, both synthetic or natural products of the ester, ether, aldehyde, ketone, alcohol, hydrocarbon, acid, carboxylic acid ester, aromatic hydrocarbon, aliphatic hydrocarbon type, saturated and/or unsaturated hydrocarbons and mixtures thereof can be used as fragrances.

As the fragrance aldehydes or fragrance ketones, all usual fragrant aldehydes and fragrant ketones can be employed which are typically used to procure a pleasant fragrant sensation. Suitable fragrant aldehydes and fragrant ketones are generally known to the person skilled in the art.

The fragrant ketones can include all ketones that can lend a desired fragrance or a sensation of freshness. Mixtures of different ketones can also be used. For example, the ketone can be selected from the group consisting of buccoxime, iso jasmone, methyl beta naphthyl ketone, moschus indanone, tonalid/moschus plus, alpha-damascone, beta-damascone, delta-damascone, iso-damascone, damascenone, damarose, methyl dihydrojasmonate, menthone, carvone, camphor, fenchone, alpha-ionone, beta-ionone, dihydro-beta-ionone, gamma-methyl so called ionone, fleuramone, dihydrojasmone, cis-jasmone, iso-E-super, methyl cedrenyl ketone or methyl cedrylone, acetophenone, methyl-acetophenone, para-methoxy-acetophenone, methyl-beta-naphthyl-ketone, benzylacetone, benzophenone, para-hydroxy-phenyl butanone, celery ketone or livescone, 6-isopropyldecahydro-2-naphthone, dimethyloctenone, freskomenth, 4-(1-ethoxyvinyl)-3,3,5,5-tetramethyl-cyclohexanone, methyl heptanone, 2-(2-(4-methyl-3-cyclohexene-1-yl)propyl)-cyclopentanone, 1-(p-Menthene-6(2)-yl)-1-propanone, 4-(4-hydroxy-3-methoxyphenyl)-2-butanone, 2-acetyl-3,3-dimethyl-norbornane, 6,7-dihydro-1,1,2,3,3-pentamethyl-4(5H)-indanone, 4-damascol, dulcinyl or cassione, gelsone, hexylone, isocyclemone E, methyl-cyclocitrone, methyl lavendel ketone, orivone, para-tert-butyl cyclohexanone, verdone, delphone, muscone, neobutenone, plicatone, veloutone, 2,4,4,7-tetramethyl-oct-6-en-3-on, tetrameran, hedione and mixtures thereof. The ketones can preferably be selected from alpha damascone, delta damascone, iso damascone, carvone, gamma-methyl-ionone, iso-E-su per, 2,4,4,7-tetramethyl-oct-6-en-3-one, benzylacetone, beta damascone, damascenone, methyl dihydrojasmonate, methyl cedrylone, hedione and mixtures thereof.

The fragrant aldehydes can be any aldehyde that produces, like the fragrant ketones, a desired fragrance or a sensation of freshness. Once again, they may be individual aldehydes or mixtures of aldehydes. Exemplary suitable aldehydes are melonal, triplal, ligustral, adoxal, anisaldehyde, cymal, ethyl vanillin, florhydral, floralozon, helional, heliotropin, hydroxycitronellal, coavone, lauric aldehyde, canthoxal, lyral, lilial, adoxal, anisaldehyde, cumal methyl-nonyl acetaldehyde, citronellal, citronellyloxy-acetaldehyde, cyclamenaldehyde, bourgeonal, p,t-bucinal, phenylacetaldehyde, undecylene aldehyde, vanillin; 2,6,10-trimethyl-9-undecenal, 3-dodecen-1-al, alpha-n-amylcinnamaldehyde, 4-methoxybenzaldehyde, benzaldehyde, 3-(4-tert-butylphenyl)-propanal, 2-methyl-3-(para-methoxyphenylpropanal), 2-methyl-4-(2,6,6-trimethyl-2(1)-cyclohexen-1-yl)butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-Isopropylbenzyaldehyde, 1,2,3,4,5,6,7,8-octahydro-8,8-dimethyl-2-naphthaldehyde, 2,4-dimethyl-3-cyclohexen-1-carboxyaldehyde, 2-methyl-3-(isopropylphenyl)propanal, decyl aldehyde, 2,6-dimethyl-5-heptenal; 4-(tricyclo[5.2.1.0(2, 6)]-decylidene-8)-butanal; octahydro-4,7-methano-1H-indenecarboxaldehyde; 3-ethoxy-4-hydroxybenzaldehyde, para-ethyl-alpha, alpha-dimethylhydrocinnamaldehyde, alpha-methyl-3,4-(methylenedioxy)-hydrocinnamaldehyde, 3,4-methylenedioxybenzaldehyde, alpha-n-hexylcinnamaldehyde, m-cymene-7-carboxaldehyde, alpha-methylphenylacetaldehyde, 7-hydroxy-3,7-dimethyl octanal, undecenal, 2,4,6-trimethyl-3-cyclohexene-1-carboxaldehyde, 4-(3)(4-methyl-3-pentenyl)-3-cyclohexene carboxaldehyde, 1-dodecanal, 2,4-dimethyl cyclohexene-3-carboxaldehyde, 4-(4-hydroxy-4-methyl-pentyl)-3-cylohexene-1-carboxaldehyde, 7-methoxy-3,7-dimethyloctan-1-al, 2-methyl undecanal, 2-methyl decanal, 1-nonanal, 1-octanal, 2,6,10-trimethyl-5,9-undecadienal, 2-methyl-3-(4-tertbutyl)propanal, 3-(4-ethylphenyl)-2,2-dimethylpropanal, 3-(4-methoxyphenyl)-2-methylpropanal, methyl-nonyl acetaldehyde, 2-phenylpropan-1-al, 3-phenylprop-2-en-1-al, 3-phenyl-2-pentylprop-2-en-1-al, 3-phenyl-2-hexylprop-2-enal, 3-(4-isopropylphenyl)-2-methylpropan-1-al, 3-(4-ethylphenyl)-2,2-dimethylpropan-1-al, 3-(4-tert-butylphenyl)-2-methyl-propanal, 3-(3,4-methylenedioxyphenyl)-2-methyl propan-1-al, 3-(4-ethyl phenyl)-2,2-dimethylpropanal, 3-(3-Isopropylphenyl)butan-1-al, 2,6-dimethylhept-5-en-1-al, dihydrocinnamaldehyde, 1-methyl-4-(4-methyl-3-pentenyl)-3-cyclohexene-1-carboxaldehyde, 5- or 6-methoxyhexahydro-4,7-methanoindane-1- or 2-carboxy aldehyde, 3,7-dimethyloctan-1-al, 1-undecanal, 10-undecen-1-al, 4-hydroxy-3-methoxybenzaldehyde, 1-methyl-3-(4-methylpentyl)-3-cyclohexenecarboxy aldehyde, 7-hydroxy-3,7-dimethyl-octanal; trans-4-decenal, 2,6-nonadienal, para-tolylacetaldehyde; 4-methylphenylacetaldehyde, 2-methyl-4-(2,6,6-trimethyl-1-cyclohexen-1-yl)-2-butenal, ortho-methoxycinnamaldehyde, 3,5,6-trimethyl-3-cyclohexene carboxaldehyde, 3,7-dimethyl-2-methylene-6-octenal, phenoxyacetaldehyde; 5,9-dimethyl-4,8-decadienal, peony aldehyde (6,10-dimethyl-3-oxa-5,9-undecadien-1-al), hexahydro-4,7-methanoindane-1-carboxaldehyde, octanal, 2-methyl octanal, alpha-methyl-4-(1-methylethyl)benzene acetaldehyde, 6,6-dimethyl-2-norpinene-2-propionaldehyde, para-methylphenoxy acetaldehyde, 2-methyl-3-phenyl-2-propen-1-al, 3,5,5-trimethylhexanal, hexahydro-8,8-dimethyl-2-naphthaldehyde, 3-propyl-bicyclo [2.2.1]-hept-5-ene-2-carbaldehyde, 9-decenal, 3-methyl-5-phenyl-1-pentanal, methylnonyl acetaldehyde, 1-p-menthene-q-carboxaldehyde, citral or mixtures thereof, lilial citral, 1-decanal, n-undecanal, n-dodecanal, florhydral, 2,4-dimethyl-3-cyclohexen-1-carboxaldehyde, 4-methoxybenzaldehyde, 3-methoxy-4-hydroxybenzaldehyde, 3-ethoxy-4-hydroxybenzaldehyde, 3,4-methylenedioxybenzaldehyde and 3,4-dimethoxybenzaldehyde and mixtures thereof.

As mentioned previously in the examples, the fragrant aldehydes and the fragrant ketones can have an aliphatic, cycloaliphatic, aromatic, ethylenically unsaturated structure or a combination of these structures. Additional heteroatoms or polycyclic structures can also be present. The structures can possess suitable substituents such as hydroxyl or amino groups.

For further suitable fragrances selected from aldehydes and ketones, reference is made to Steffen Arctander, Aroma Chemicals Vol. 1, ISBN: 0-931710-37-5, Aroma Chemicals Vol. 2: 0-931710-38-3, published 1960 and 1969 respectively, reprinted 2000.

Suitable odoriferous compounds of the ester type are, for example, benzyl acetate, phenoxyethyl isobutyrate, p-tert.-butylcyclohexyl acetate, linalyl acetate, dimethylbenzyl carbinyl acetate (DMBCA), phenylethyl acetate, benzyl acetate, ethylmethylphenyl glycinate, allylcyclohexyl propionate, styrallyl propionate, benzyl salicylate, cyclohexyl salicylate, floramate, melusate and jasmecyclate. Odoriferous compounds of the hydrocarbon type are e.g. terpenes such as limonene and pinene. Suitable fragrances of the ether type are for example benzyl ethyl ether and ambroxane. Exemplary fragrant alcohols are 10-undecen-1-ol, 2,6-dimethylheptan-2-ol, 2-methylbutanol, 2-methylpentanol, 2-phenoxyethanol, 2-phenylpropanol, 2-tert-butylcyclohexanol, 3,5,5-trimethylcyclohexanol, 3-hexanol, 3-methyl-5-phenyl pentanol, 3-octanol, 1-octen-3-ol, 3-phenylpropanol, 4-heptenol, 4-isopropylcyclohexanol, 4-tert-butylcyclohexanol, 6,8-dimethyl-2-nonanol, 6-nonen-1-ol, 9-decen-1-ol, alpha-methyl benzyl alcohol, alpha-terpineol, amyl salicylate, benzyl alcohol, benzyl salicylate, beta-terpineol, butyl salicylate, citronellol, cyclohexyl salicylate, decanol, dihydromyrcenol, dimethylbenzyl carbinol, dimethylheptanol, dimethyloctanol, ethyl salicylate, ethylvanilin, anethol, eugenol, geraniol, heptanol, hexyl salicylate, isoborneol, isoeugenol, isopulegol, linalool, menthol, myrtenol, n-hexanol, nerol, nonanol, octanol, para-menthan-7-ol, phenylethyl alcohol, phenol, phenyl salicylate, tetrahydrogeraniol, tetrahydrolinalool, thymol, trans-2-cis-6-nonadienol, trans-2-nonen-1-ol, trans-2-octenol, undecanol, vanillin, cinnamyl alcohol, wherein if a plurality of fragrant alcohols is present, they can be selected independently of one another.

Fragrances or perfume oils may also be natural perfume mixtures as can be obtained from vegetal sources, for example pine, citrus, jasmine, patchouli, rose or ylang-ylang oil. Muscatel sage oil, chamomile oil, oil of cloves, balm mint oil, mint oil, cinnamon leaf oil, linden flower oil, juniper oil, vetiver oil, olibanum oil, galbanum oil and labdanum oil as well as orange leaf oil, orange flower oil, orange peel oil and sandal wood oil, ethereal oils such as angelica root oil, aniseed, arnica flower oil, basil oil, oil of bay, champaca leaf oil, silver fir oil, silver fir cone oil, elemi oil, eucalyptus oil, fennel oil, pine-needle oil, galbanum oil, geranium oil, ginger grass oil, guaiacol, gurjun balsam oil, helichrysum oil, ho oil, ginger oil, irisol, cajeput oil, calamus oil, camomile oil, camphor oil, kanaga oil, cardamom oil, cassia oil, pine needle oil, balsams copaiba oil, coriander oil, spearmint oil, caraway oil, cumin oil, lavender oil, lemon grass oil, lime oil, mandarin oil, melissa oil, musk seed oil, oil of myrrh, oil of cloves, oil of orange flowers, niauli oil, olibanum oil, origano oil, palmarosa oil, patchuli oil, Peru balsam oil, petitgrain oil, pepper oil, peppermint oil, allspice oil, pine oil, rose oil, rosemary oil, sandal wood oil, celery oil, oil of spike lavender, star anise oil, terpentine, thuja oil, oil of thyme, verbena oil, vetiver oil, juniper berry oil, oil of wormwood, oil of wintergreen, ylang ylang oil, hyssop oil, cinnamon oil, cinnamon leaf oil, citronellol, lemon oil as well as Cyprus, oil are likewise suitable.

The total quantity of the at least one fragrance in the inventive agent for textile or surface treatment is preferably between 0.01 and 5 wt. %, particularly preferably between 0.1 and 3 wt. % and quite particularly preferably between 0.5 and 2 wt. % based on the total quantity of the composition. Mixtures of various fragrances (from the various fragrances cited above), which together produce an attractive fragrant note, are preferably used. In this case the total quantity of the at least one fragrance is the quantity of all fragrances together in the mixture based on the total quantity of the composition.

Besides the inventively already mentioned esterquats as fabric softening active principles, there exist additional softening components that can also be used in the compositions according to the invention. These include for example quaternary ammonium compounds such as monoalk(en)yltrimethylammonium compounds, dialk(en)yldimethylammonium compounds, mono-, di- or triesters of fatty acids with alkanolamines.

Suitable examples of quaternary ammonium compounds are shown for example in the Formulas (II) and (III):

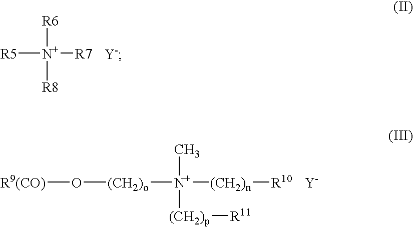

wherein in (II) R₅ stands for an alkyl group having 12 to 24 carbon atoms, R₆ stands for a saturated C1-C4 alkyl or hydroxyalkyl group, R₇ and R₈ are either equal to R₅ or R₆ or stand for an aromatic group. Y stands either for a halide ion, methosulfate ion, methophosphate ion or phosphate ion as well as their mixtures. Exemplary cationic compounds of Formula (II) are monotallowtrimethylammonium chloride, monostearyl trimethylammonium chloride, didecyldimethyl ammonium chloride, ditallowedimethylammonium chloride or dihexadecylammonium chloride.

Compounds of Formula (III), (IV) and (V) are so-called esterquats. Esterquats are characterized by their outstanding biodegradability. In Formula (III), R₉ stands for an aliphatic alk(en)yl group containing 12 to 22 carbon atoms with 0, 1, 2 or 3 double bonds and/or optionally with substituents; R₁₀ stands for H, OH or O(CO)R₁₂, R₁₁ independently of R₁₀ stands for H, OH or O(CO)R₁₃, wherein R₁₂ and R₁₃, independently of each other, each stand for an aliphatic alk(en)yl group having 12 to 22 carbon atoms with 0, 1, 2 or 3 double bonds, n, p and o independently of each other can each have the value 1, 2 or 3. Y can be either a halide ion, methosulfate ion, methophosphate ion or phosphate ion as well as mixtures of these anions. Compounds are preferred in which R₁₀ represents the group O(CO)R₁₂. Compounds are particularly preferred in which R₁₀ represents the group O(CO)R₁₂ and R₉ and R₁₂ are alk(en)yl groups with 16 to 18 carbon atoms. Particularly preferred are compounds in which R₆ stands moreover for OH. Examples of compounds of Formula (III) are methyl-N-(2-hydroxyethyl)-N,N-di(tallowacyloxyethyl)ammonium methosulfate, bis(palmitoyloxyethyl)-hydroxyethyl-methyl-ammonium methosulfate, 1,2-bis-[tallowacyloxy]-3-trimethylammoniumpropane chloride or methyl-N,N-bis(stearoyloxyethyl)-N-(2-hydroxyethyl)ammonium methosulfate.

When quaternized compounds of Formula (III) are used that have unsaturated alkyl chains, the acyl groups are preferred, whose corresponding fatty acids have an iodine number between 1 and 100, preferably between 5 and 80 and more particularly between 10 and 60 and in particular between 15 and 45, which have a cis/trans isomer ratio (in wt. %) of greater than 30:70, preferably greater than 50:50 and particularly equal to or greater than 60:40. Commercial examples are the methylhydroxyalkyl-dialkoyloxyalkylammonium methosulfates marketed by Stepan under the trade name Stepantexe or products from Cognis known under the trade name Dehyquart® or the products manufactured by Degussa known under the name Rewoquat® or products from Kao known as Tetranyl®. Further preferred compounds are the diesterquats of Formula (IV), which are available under the names Rewoquat® W 222 LM or CR 3099.

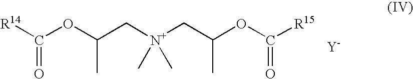

R₁₄ und R₁₅ stand, independently of each other, each for an aliphatic group having 12 to 22 carbon atoms with 0, 1, 2 or 3 double bonds.

Instead of the ester group O(CO)R, wherein R stands for a long chain alk(en)yl group, softening compounds can be added that possess the following groups: RO(CO), N(CO)R or RN(CO), wherein the N(CO)R groups are preferred among these groups.

In addition to the above-described quaternary compounds, other compounds can also be added as the softening components, such as for example quaternary imidazolinium compounds of Formula (V),

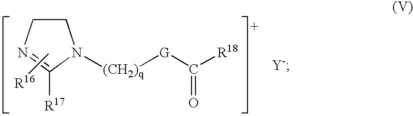

wherein R₁₆ stands for a saturated alkyl group with 1 to 4 carbon atoms, R₁₇ and R₁₈, independently of one other, each stand for an aliphatic, saturated or unsaturated alkyl group with 12 to 18 carbon atoms, R₁₇ can alternatively also stand for O(CO)R₁₉, wherein R₁₉ means an aliphatic, saturated or unsaturated alkyl group with 12 to 18 carbon atoms, and G means an NH group or oxygen and Y is an anion; q can assume whole number values between 1 and 4.

Further particularly preferred softening compounds are described by Formula (VI),

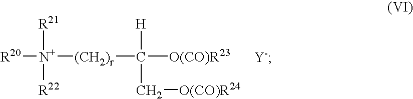

wherein R₂₀, R₂₁ and R₂₂ independently of one another stand for a C₁₋₄ alkyl, alkenyl or hydroxyalkyl group, R₂₃ and R₂₄, each independently selected, represents a C₈₋₂₈ alkyl group, Y is an anion and r is a number between 0 and 5. A preferred example of a cationic precipitation auxiliary according to Formula (VI) is 2,3-bis[tallowacyloxy]-3-trimethylammonium propane chloride.

Quaternized protein hydrolyzates or protonated amines represent further inventively usable softening components.

In addition, cationic polymers are also suitable softening components. Suitable cationic polymers include the polyquaternium polymers such as those in the CTFA Cosmetic Ingredient Dictionary (The Cosmetic, Toiletry und Fragrance, Inc., 1997), particularly those polyquaternium-6, polyquaternium-7, polyquaternium-10 polymers also described as Merquats (Polymer JR, LR and KG series from Amerchol), polyquaternium-4-copolymers, such as graft copolymers with a cellulosic backbone and quaternary ammonium groups that are bonded through allyldimethylammonium chloride, cationic cellulose derivatives like cationic guar, such as guarhydroxypropyltriammonium chloride, and similar quaternized guar derivatives (e.g. Cosmedia Guar, manufactured by Cognis or the Jaguar series from Rhodia), cationic quaternary sugar derivatives (cationic alkyl polyglucosides), e.g. the commercial product Glucquate 100, according to CTFA nomenclature a “Lauryl Methyl Gluceth-10 Hydroxypropyl Dimonium Chloride”, copolymers of PVP and dimethylamino methacrylate, copolymers of vinyl imidazole and vinyl pyrrolidone, amino silicone polymers and copolymers.

Polyquaternized polymers (e.g. Luviquat® Care from BASF) and also cationic biopolymers based on chitin and its derivatives, for example the polymer obtained under the trade name Chitosan® (manufacturer: Cognis) can also be employed.

Some of the cited cationic polymers additionally exhibit skin care and/or textile care properties.

Compounds of Formula (VII) are likewise usable,

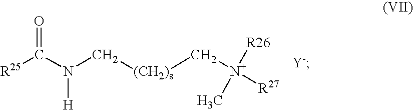

R₂₅ can be an aliphatic alk(en)yl group having 12 to 22 carbon atoms with 0, 1, 2 or 3 double bonds, s can assume values between 0 and 5. R₂₆ and R₂₇ stand, independently of one another, each for H, C1-4 alkyl or hydroxyalkyl and Y is an anion.

Further suitable softening components include protonated or quaternized polyamines.

Alkylated quaternary ammonium compounds having at least one alkyl chain interrupted by an ester group and/or an amido group are particularly preferred. N-methyl-N-(2-hydroxyethyl)-N,N-(ditallowacyloxyethyl)ammonium methosulfate or bis-(palmitoyloxyethyl)-hydroxyethyl-methyl-ammonium methosulfate are quite particularly preferred.

The agents for textile treatment in the form of rinse aids can also comprise non-ionic softening components such as principally polyoxyalkylene glycerol alkanoates, polybutylenes, long chain fatty acids, ethoxylated fatty acid ethanolamides, alkyl polyglucosides, particularly mono, di and triesters of sorbitol, and fatty acid esters of polycarboxylic acids.

The inventive rinse aid preferably comprises the softening component as the agent for treating textiles in an amount of typically 0.1 to 80 wt. %, normally 1 to 40 wt. %, preferably 2 to 20 wt. % and particularly 3 to 15 wt. %, each based on the total quantity of the agent for treating textiles.

The agents for treating textiles in the form of rinse aids can optionally comprise one or more non-ionic surfactants as additional components, wherein those surfactants can be added that are also normally employed in detergents.

The agent for treating textiles can be a detergent, rinse aid, softening detergent or a detergent auxiliary, wherein said agent can be solid or liquid and liquid detergents are preferred.

In addition to the fragrances and OH group-containing esterquats, the inventive agents in the form of detergents preferably comprise surfactant(s), wherein anionic, cationic, non-ionic, zwitterionic, gemini and/or amphoteric surfactants can be employed.

Mixtures of non-ionic and cationic surfactants are preferred from the technical viewpoint. The total surfactant content of a liquid detergent is preferably below 40 wt. % and particularly preferably below 35 wt. %, based on the total liquid detergent.

Preferred non-ionic surfactants are alkoxylated, advantageously ethoxylated, particularly primary alcohols preferably containing 8 to 18 carbon atoms and, on average, 1 to 12 moles of ethylene oxide (EO) per mole of alcohol, in which the alcohol group may be linear or, preferably, methyl-branched in the 2-position or may contain e.g. linear and methyl-branched groups in the form of the mixtures typically present in oxo alcohol groups. In particular, however, alcohol ethoxylates with linear alcohol groups of natural origin with 12 to 18 carbon atoms, e.g. from coco-, palm-, tallow- or oleyl alcohol, and an average of 2 to 8 EO per mole alcohol are preferred. Exemplary preferred ethoxylated alcohols include C12-14 alcohols with 3 EO, 4EO or 7EO, C9-11 alcohol with 7 EO, C13-15 alcohols with 3 EO, 5 EO, 7 EO or 8 EO, C12-18 alcohols with 3EO, 5EO or 7EO and mixtures thereof, as well as mixtures of C12-14 alcohols with 3 EO and C12-18 alcohols with 7 EO. The cited degrees of ethoxylation constitute statistically average values that can be a whole or a fractional number for a specific product. Preferred alcohol ethoxylates have a narrowed homolog distribution (narrow range ethoxylates, NRE). In addition to these non-ionic surfactants, fatty alcohols with more than 12 EO can also be used. Examples of these are tallow fatty alcohol with 14 EO, 25 EO, 30 EO or 40 EO. Also, non-ionic surfactants that comprise the EO— and PO groups together in the molecule are employable according to the invention. Here, block copolymers with EO-PO blocks or PO-EO blocks can be added, but also EO—PO-EO copolymers or PO-EO-PO copolymers. Of course, mixed alkoxylated non-ionic surfactants can also be used, in which EO— and PO— units are not in blocks but rather distributed statistically. Such products can be obtained by the simultaneous action of ethylene oxide and propylene oxide on fatty alcohols.

Furthermore, as additional non-ionic surfactants, alkyl glycosides that satisfy the general Formula RO(G)ₓ can be added, where R means a primary linear or methyl-branched, particularly 2-methyl-branched, aliphatic group containing 8 to 22 and preferably 12 to 18 carbon atoms and G stands for a glucose unit containing 5 or 6 carbon atoms, preferably glucose. The degree of oligomerization x, which defines the distribution of monoglycosides and oligoglycosides, is any number between 1 and 10, preferably between 1.2 and 1.4. Alkyl glycosides are known, mild surfactants and are therefore preferably employed in the surfactant mixture.

Another class of preferred non-ionic surfactants which may be used, either as the sole non-ionic surfactant or in combination with other non-ionic surfactants are alkoxylated, preferably ethoxylated or ethoxylated and propoxylated fatty acid alkyl esters preferably containing 1 to 4 carbon atoms in the alkyl chain, in particular fatty acid methyl esters.

Non-ionic surfactants of the amine oxide type, for example N-cocoalkyl-N,N-dimethylamine oxide and N-tallow alkyl-N,N-dihydroxyethylamine oxide, and the fatty acid alkanolamides may also be suitable. The quantity in which these non-ionic surfactants are used is preferably no more than the quantity in which the ethoxylated fatty alcohols are used and, particularly no more than half that quantity.

Other suitable surfactants are polyhydroxyfatty acid amides corresponding to the Formula (VIII),

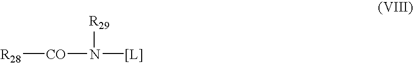

in which R₂₈CO stands for an aliphatic acyl group with to 6 to 22 carbon atoms, R₂₉ for hydrogen, an alkyl or hydroxyalkyl group with to 1 to 4 carbon atoms and [L] for a linear or branched polyhydroxyalkyl group with 3 to 10 carbon atoms and 3 to 10 hydroxyl groups. The polyhydroxyfatty acid amides are known substances, which may normally be obtained by reductive amination of a reducing sugar with ammonia, an alkylamine or an alkanolamine and subsequent acylation with a fatty acid, a fatty acid alkyl ester or a fatty acid chloride.

The group of polyhydroxyfatty acid amides also includes compounds corresponding to the Formula (IX),

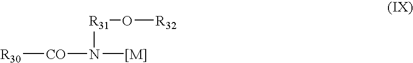

in which R₃₀ is a linear or branched alkyl or alkenyl group containing 7 to 12 carbon atoms, R₃₁ is a linear, branched or cyclic alkyl group or an aryl group containing 2 to 8 carbon atoms and R₃₂ is a linear, branched or cyclic alkyl group or an aryl group or an oxyalkyl group containing 1 to 8 carbon atoms, C1-4 alkyl or phenyl groups being preferred, and [M] is a linear polyhydroxyalkyl group, of which the alkyl chain is substituted by at least two hydroxyl groups, or alkoxylated, preferably ethoxylated or propoxylated derivatives of that group.

[M] is preferably obtained by reductive amination of a sugar, for example glucose, fructose, maltose, lactose, galactose, mannose or xylose. The N-alkoxy- or N-aryloxy-substituted compounds may then be converted into the required polyhydroxyfatty acid amides by reaction with fatty acid methyl esters in the presence of an alkoxide as catalyst.

The content of non-ionic surfactants in the agents for treating textiles in the form a liquid detergent is preferably 5 to 30 wt. %, advantageously 7 to 20 wt. % and particularly 9 to 15 wt. %, in each case based on the total agent for treating textiles.

Exemplary suitable anionic surfactants are those of the sulfonate and sulfate type. Suitable surfactants of the sulfonate type are, advantageously C9-13 alkylbenzene sulfonates, olefin sulfonates, i.e. mixtures of alkene- and hydroxyalkane sulfonates and disulfonates, as are obtained, for example, from C12-18 monoolefins having a terminal or internal double bond, by sulfonation with gaseous sulfur trioxide and subsequent alkaline or acidic hydrolysis of the sulfonation products. Those alkane sulfonates, obtained from C12-18 alkanes by sulfochlorination or sulfoxidation, for example, with subsequent hydrolysis or neutralization, are also suitable. The esters of α-sulfofatty acids (ester sulfonates), e.g. the α-sulfonated methyl esters of hydrogenated coco-, palm nut- or tallow acids are likewise suitable.

Further suitable anionic surfactants are sulfated fatty acid esters of glycerine. Fatty acid glycerine esters are understood to include the mono-, di- and triesters and also their mixtures, such as those obtained by the esterification of a monoglycerine with 1 to 3 moles fatty acid or by the transesterification of triglycerides with 0.3 to 2 moles glycerine. Preferred sulfated fatty acid esters of glycerol in this case are the sulfated products of saturated fatty acids with 6 to 22 carbon atoms, for example caproic acid, caprylic acid, capric acid, myristic acid, lauric acid, palmitic acid, stearic acid or behenic acid.

Preferred alk(en)yl sulfates are the alkali metal and especially the sodium salts of the sulfuric acid half-esters derived from the C₁₂-C₁₈ fatty alcohols, for example from coconut butter alcohol, tallow alcohol, lauryl, myristyl, cetyl or stearyl alcohol or from C10-C20 oxo alcohols and those half-esters of secondary alcohols of these chain lengths. Additionally preferred are alk(en)yl sulfates of the said chain lengths, which contain a synthetic, straight-chained alkyl group produced on a petrochemical basis and which show similar degradation behaviour to the suitable compounds based on fat chemical raw materials. The C12-C16 alkyl sulfates and C12-C15 alkyl sulfates and C14-C15 alkyl sulfates are preferred on the grounds of laundry performance. 2,3-Alkyl sulfates, which can be obtained from the Shell Oil Company under the trade name DAN®, are also suitable anionic surfactants.

Sulfuric acid mono-esters derived from straight-chain or branched C₇₋₂₁ alcohols ethoxylated with 1 to 6 moles ethylene oxide are also suitable, for example 2-methyl-branched C9-11 alcohols with an average of 3.5 mole ethylene oxide (EO) or C12-18 fatty alcohols with 1 to 4 EO. Due to their high foaming performance, they are only used in fairly small quantities in cleaning compositions, for example in amounts of 1 to 5% by weight.

Other suitable anionic surfactants are also the salts of alkylsulfosuccinic acid, which are also referred to as sulfosuccinates or esters of sulfosuccinic acid, and the monoesters and/or di-esters of sulfosuccinic acid with alcohols, preferably fatty alcohols and especially ethoxylated fatty alcohols. Preferred sulfosuccinates comprise C8-18 fatty alcohol groups or mixtures of them. Especially preferred sulfosuccinates comprise a fatty alcohol group derived from ethoxylated fatty alcohols and may be considered as non-ionic surfactants (see description below). Once again the particularly preferred sulfosuccinates are those, whose fatty alcohol groups are derived from ethoxylated fatty alcohols with narrow range homolog distribution. It is also possible to use alk(en)ylsuccinic acids with preferably 8 to 18 carbon atoms in the alk(en)yl chain, or salts thereof.

Particularly preferred anionic surfactants are soaps. Saturated and unsaturated fatty acid soaps are suitable, such as the salts of lauric acid, myristic acid, palmitic acid, stearic acid, (hydrogenated) erucic acid and behenic acid, and especially soap mixtures derived from natural fatty acids such as coconut oil fatty acid, palm kernel oil fatty acid, olive oil fatty acid or tallow fatty acid.

Anionic surfactants, including soaps may be in the form of their sodium, potassium or ammonium salts or as soluble salts of organic bases, such as mono-, di- or triethanolamine. Preferably, the anionic surfactants are in the form of their sodium or potassium salts, especially in the form of the sodium salts.

The content of anionic surfactants in the preferred agents for treating textiles in the form of detergents is preferably 2 to 30 wt. %, preferably 4 to 25 wt. % and particularly 5 to 22 wt. %, in each case based on the total agent for treating textiles.

The so-called gemini surfactants can be considered as further surfactants. Generally speaking, such compounds are understood to mean compounds that have two hydrophilic groups and two hydrophobic groups per molecule. As a rule, these groups are separated from one another by a “spacer”. The spacer is usually a hydrocarbon chain that is intended to be long enough such that the hydrophilic groups are a sufficient distance apart to be able to act independently of one another. These types of surfactants are generally characterized by an unusually low critical micelle concentration and the ability to strongly reduce the surface tension of water. In exceptional cases, however, not only dimeric but also trimeric surfactants are meant by the term gemini surfactants. Suitable gemini surfactants are, for example, sulfated hydroxy mixed ethers according to the German Patent application DE-A-43 21 022 or dimer alcohol bis- and trimer alcohol tris sulfates and ether sulfates according to the international patent application WO-A-96/23768. Blocked end group dimeric and trimeric mixed ethers according to the German Patent application DE-A-195 13 391 are especially characterized by their bifunctionality and multifunctionality. Thus, the cited blocked end group surfactants possess good wetting properties and are therefore poor foamers, such that they are particularly suited for use in automatic washing or cleaning processes. However, gemini polyhydroxyfatty acid amides or poly-polyhydroxyfatty acid amides, such as those described in the international Patent applications WO-A-95/19953, WO-A-95/19954 and WO-A-95/19955 can also be used. Further preferred agents for treating textiles include softening detergents and detergent auxiliaries.

Softening detergents are understood to mean agents that simultaneously clean and condition the treated textiles. For that purpose they also comprise a softening component in addition to the surfactants. The softening component can be a cationic or non-ionic softening component as described above as well as a softening clay (for example bentonite).

Detergent auxiliaries are added prior to washing for a specific pre-treatment of the washing for stains or heavy soiling. The detergent auxiliaries include for example pre-treatment agents, soaking agents, color run removers and stain remover.

It is further preferred that the agent additionally comprises usual ingredients of agents for treating textiles or surfaces. In addition to the surfactants and/or softening compounds, the agents for treating textiles can comprise additional ingredients that further improve the application performance and/or aesthetic properties of the agent for treating textiles. In the context of the present invention, preferred agents for treating textiles can additionally comprise one or a plurality of materials from the group of builders, bleaches, bleach activators, enzymes, electrolytes, non-aqueous solvents, pH adjustors, perfumes, perfume carriers, fluorescent agents, dyes, hydrotropes, foam inhibitors, silicone oils, anti-redeposition agents, optical brighteners, graying inhibitors, laddering retardants, anti-crease agents, color transfer inhibitors, antimicrobials, germicides, fungicides, antioxidants, preservatives, corrosion inhibitors, antistats, bittering agents, ironing aids, water-repellents and impregnation agents, swelling and non-skid agents, neutral filling salts and UV-absorbers.

Silicates, aluminum silicates (particularly zeolites), carbonates, salts of organic di- and polycarboxylic acids as well as mixtures of these materials can be particularly cited as builders that are comprised in the agents for treating textiles.

Suitable crystalline, layered sodium silicates correspond to the general formula NaMSiₓO₂ₓ₊₁.H₂O, wherein M is sodium or hydrogen, x is a number from 1.9 to 4 and y is a number from 0 to 20, preferred values for x being 2, 3 or 4. Preferred crystalline layered silicates of the given formula are those in which M stands for sodium and x assumes the values 2 or 3. Both β- and δ-sodium disilicates Na₂Si₂O₅yH₂O are particularly preferred.

Other useful builders are amorphous sodium silicates with a modulus (Na₂O:SiO₂ ratio) of 1:2 to 1:3.3, preferably 1:2 to 1:2.8 and more preferably 1:2 to 1:2.6, which dissolve with a delay and exhibit multiple wash cycle properties. The delay in dissolution compared with conventional amorphous sodium silicates can have been obtained in various ways, for example by surface treatment, compounding, compressing/compacting or by over-drying. In the context of this invention, the term “amorphous” also means “X-ray amorphous”. In other words, the silicates do not produce any of the sharp X-ray reflections typical of crystalline substances, but at best one or more maxima of the scattered X-radiation, which have a width of several degrees of the diffraction angle. However, particularly good builder properties may even be achieved where the silicate particles produce indistinct or even sharp diffraction maxima in electron diffraction experiments. This can be interpreted to mean that the products have microcrystalline regions between 10 and a few hundred nm in size, values of up to at most 50 nm and especially up to at most 20 nm being preferred. Compacted/densified amorphous silicates, compounded amorphous silicates and over dried X-ray-amorphous silicates are particularly preferred.

Of the suitable fine crystalline, synthetic zeolites containing bound water, zeolite A and/or P are preferred. Zeolite MAP® (commercial product of the Crosfield company), is particularly preferred as the zeolite P. However, zeolite X and mixtures of A, X and/or P are also suitable. Commercially available and preferably used in the context of the present invention is, for example, also a co-crystallizate of zeolite X and zeolite A (ca. 80 wt. % zeolite X), which is marketed by the SASOL Company under the trade name VEGOBOND AX® and which can be described by the Formula

nNa2O.(1-n)K2O.Al2O3.(2-2.5)SiO2.(3.5-5.5)H2O

n=0.90-1.0

The zeolite can be employed as the spray-dried powder or also as the non-dried, still moist from its manufacture, stabilized suspension. For the case where the zeolite is added as a suspension, this can comprise small amounts of non-ionic surfactants as stabilizers, for example 1 to 3 wt. %, based on the zeolite, of ethoxylated C12-C18 fatty alcohols with 2 to 5 ethylene oxide groups, C12-C14 fatty alcohols with 4 to 5 ethylene oxide groups or ethoxylated isotridecanols. Suitable zeolites have a mean particle size of less than 10 μm (volume distribution, as measured by the Coulter Counter Method) and contain preferably 18 to 22% by weight and more preferably 20 to 22% by weight of bound water.

Naturally, the generally known phosphates can also be added as builders, in so far that their use should not be avoided on ecological grounds. The sodium salts of the orthophosphates, the pyrophosphates and especially the tripolyphosphates are particularly suitable.

Organic builders that can be present in the agent for treating textiles include polycarboxylate polymers such as polyacrylates and acrylic acid/maleic acid copolymers, polyaspartates and monomeric polycarboxylates such as citrates, gluconates, succinates or malonates, which are preferably added as the sodium salts.

Among the compounds, which serve as bleaching agents and liberate H₂O₂ in water, sodium perborate tetrahydrate and sodium perborate monohydrate are of particular importance. Examples of additional bleaching agents that may be employed are sodium percarbonate, peroxypyrophosphates, citrate perhydrates and H₂O₂-liberating peracidic salts or peracids, such as perbenzoates, peroxyphthalates, diperoxyazelaic acid, phthaloimino peracid or diperoxydodecanedioic acid.

The detergents and cleaning compositions can comprise bleach activators in order to achieve an improved bleaching action for washing temperatures of 60° C. and below. Bleach activators, which can be used, are compounds which, under perhydrolysis conditions, yield aliphatic peroxycarboxylic acids having preferably 1 to 10 carbon atoms, in particular 2 to 4 carbon atoms, and/or optionally substituted perbenzoic acid. Substances, which carry O-acyl and/or N-acyl groups of said number of carbon atoms and/or optionally substituted benzoyl groups, are suitable. Preference is given to polyacylated alkylene diamines, in particular tetraacetyl ethylenediamine (TAED), acylated triazine derivatives, in particular 1,5-diacetyl-2,4-dioxohexahydro-1,3,5-triazine (DADHT), acylated glycolurils, in particular tetraacetyl glycoluril (TAGU), N-acylimides, in particular N-nonanoyl succinimide (NOSI), acylated phenol sulfonates, in particular n-nonanoyl- or isononanoyloxybenzene sulfonate (n- or iso-NOBS), carboxylic acid anhydrides, in particular phthalic anhydride, acylated polyhydric alcohols, in particular triacetin, ethylene glycol diacetate and 2,5-diacetoxy-2,5-dihydrofuran.

In addition to, or instead of the conventional bleach activators, so-called bleach catalysts may also be incorporated into the agent for treating textiles. These substances are bleach-boosting transition metal salts or transition metal complexes such as, for example, manganese-, iron-, cobalt-, ruthenium- or molybdenum-salen or -carbonyl complexes. Manganese, iron, cobalt, ruthenium, molybdenum, titanium, vanadium and copper complexes with nitrogen-containing tripod ligands and cobalt-, iron-, copper- and ruthenium-amine complexes may also be used as bleach catalysts.

A liquid agent for treating textiles can comprise a thickener. The thickener can include, for example a polyacrylate-thickener, Xanthane gum, gellan gum, guar nut flour, alginate, carrageenan, carboxymethylcellulose, bentonite, wellan gum, locust bean flour, agar-agar, traganth, gummi arabicum, pectins, polyoses, starches, dextrins, gelatines and casein. Modified natural products, such as modified starches and celluloses, examples being carboxymethyl cellulose and other cellulose ethers, hydroxyethyl and hydroxypropyl cellulose as well as bean flour ether, can also be employed as the thickener.

The polyacrylic and polymethacrylic thickeners include, for example, the high molecular weight homopolymers of acrylic acid, crosslinked with a polyalkenyl polyether, in particular an allyl ether of saccharose, pentaerythritol or propylene (INCI name according to the “International Dictionary of Cosmetic Ingredients” of The Cosmetic, Toiletry and Fragrance Association (CTFA): Carbomer), which are also called carboxyvinyl polymers. Such polyacrylic acids are available inter alia from 3V Sigma Company under the trade name Polygel®, e.g. Polygel DA, and from the B.F. Goodrich Company under the trade name Carbopol®, e.g. Carbopol 940 (molecular weight ca. 4 000 000), Carbopol 941 (molecular weight ca. 1,250,000 250 000) or Carbopol 3,000,000 (molecular weight ca. 3 000 000)). In addition, the following acrylic acid copolymers are included: (i) copolymers of two or more monomers from the group of acrylic acid, methacrylic acid and their simple esters, preferably formed with C₁₋₄ alcohols, (INCI acrylate copolymer), to which belong, for example, the copolymers of methacrylic acid, butyl acrylate and methyl methacrylate (CAS number according to Chemical Abstracts Service: 25035-69-2) or of butyl acrylate and methyl methacrylate (CAS 25852-37-3) and which are available, for example, from Rohm & Haas under the trade names Aculyne and Acusol®, and from Degussa (Goldschmidt) under the trade names Tego® Polymer, e.g. the anionic non-associative polymers Aculyn 22, Aculyn 28, Aculyn 33 (crosslinked), Acusol 810, Acusol 820, Acusol 823 and Acusol 830 (CAS 25852-37-3); (ii) crosslinked high molecular weight acrylic acid copolymers that include, for example copolymers of C10-30 alkyl acrylates and one or more monomers from the group of acrylic acid, methacrylic acid and their simple esters, preferably formed with C1-4 alcohols, which are crosslinked with an allyl ether of saccharose or of pentaerythritol (INCI Acrylates/C10-30 alkyl acrylate crosspolymer) and which are available from the B.F. Goodrich Company under the trade name Carbopol®, e.g. the hydrophobized Carbopol ETD 2623 and Carbopol 1382 (INCI Acrylates/C10-30 Alkyl Acrylate Crosspolymer) as well as Carbopol Aqua 30 (previously Carbopol EX 473).

A further preferred employable polymeric thickener is Xanthane gum, a microbial anionic heteropolysaccharide that is produced under aerobic conditions by Xanthomonas campestris and some other species, and which has a molecular weight of 2 to 15 million Dalton. Xanthane is formed from a chain of linked β-1,4-glucose (cellulose) with side chains. The composition of the sub-groups consists of glucose, mannose, glucuronic acid, acetate and Pyruvate, wherein the number of pyruvate units determines the viscosity of the Xanthane gum.

In particular, a fatty alcohol can also be considered as a thickener. Fatty alcohols can be branched or unbranched and be of natural origin or of petrochemical origin. Preferred fatty alcohols have a carbon chain length of 10 to 20 carbon atoms, preferably 12 to 18. Mixtures of different carbon chain lengths are preferred such as tallow fatty alcohol or coco fatty alcohol. Examples are Lorol® Spezial (C12-14-ROH) or Lorol® Technisch (C12-18-ROH) (both from Cognis).

Preferred liquid agents for treating textiles comprise 0.01 to 3 wt. % and preferably 0.1 to 1 wt. % thickener, based on the total agent for treating textiles. The amount of added thickener depends on the type of thickener and the desired degree of thickening.

The agent for treating textiles can comprise encapsulated enzymes and/or enzymes directly in the agent for treating textiles. Suitable enzymes are, in particular, those from the classes of hydrolases, such as proteases, esterases, lipases or lipolytic enzymes, amylases, cellulases or other glycosyl hydrolases, hemicellulases, cutinases, β-glucanases, oxidases, peroxidases, perhydrolases and/or laccases and mixtures of the cited enzymes. In the wash, all these hydrolases contribute to the removal of stains such as protein, fat or starchy stains and against graying. Moreover, cellulases and other glycosyl hydrolases can contribute to increased softness of the textile and to color retention by removing pilling and micro fibrils. Oxireductases can also be added for bleaching or for reducing color transfer. Enzymatic active materials obtained from bacterial sources or fungi such as bacillus subtilis, bacillus licheniformis, streptomyceus griseus and humicola insolens are particularly well suited. Proteases of the subtilisin type and particularly proteases that are obtained from bacillus lentus, are preferably used. Here, mixtures of enzymes are of particular interest, for example proteases and amylases or proteases and lipases or lipolytic enzymes or proteases and cellulases or cellulases and lipases or lipolytic enzymes or proteases, amylases and lipases or lipolytic enzymes or proteases, lipases or lipolytic enzymes and cellulases, in particular, however proteases and/or lipase-containing mixtures or mixtures with lipolytic enzymes. Examples of such lipolytic enzymes are the known cutinases. Peroxidases or oxidases have also proved to be suitable in certain cases. The suitable amylases particularly include α-amylases, iso-amylases, pullulanases and pectinases. Cellobiohydrolases, endoglucanases and β-glucosidases or mixtures thereof, which are also known as cellobiases are preferred cellulases. As the different cellulase types differ in their CMCase- and avicelase activities, the required activities can be adjusted by means of controlled mixtures of the cellulases.

The enzymes can be adsorbed on-carriers in order to protect them against premature decomposition. The content of the enzymes, liquid enzyme formulation(s) or enzyme granules directly in the agent for treating textiles can be, for example, about 0.01 to 5% by weight and is preferably 0.12 to about 2.5% by weight.

However, for example for specific agents for treating textiles for consumers with allergies and/or sensitive skin, it can also be preferred that the agent for treating textiles does not comprise enzymes.

A large number of the most varied salts from the group of the inorganic salts can be employed as the electrolytes. Preferred cations are the alkali metal and alkaline earth metals, preferred anions are the halides and sulfates. The addition of NaCl or MgCl₂ to the agents for treating textiles is preferred from the industrial manufacturing point of view. The content of electrolytes in the agents for treating textiles normally ranges from 0.1 to 5 wt. %.

Non-aqueous solvents that can be added to the liquid agents for treating textiles originate for example from the group of mono- or polyvalent alcohols, alkanolamines or glycol ethers, in so far that they are miscible with water in the defined concentrations. Preferably, the solvents are selected from ethanol, n- or i-propanol, butanols, glycol, propane diol or butane diol, glycerine, diglycol, propyl diglycol or butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl-, -ethyl- or -propyl ether, dipropylene glycol methyl-, or -ethyl ether, diisopropylene glycol methyl-, or -ethyl ether, methoxy-, ethoxy- or butoxy triglycol, 1-butoxyethoxy-2-propanol, 3-methyl-3-methoxybutanol, propylene glycol t-butyl ether as well as mixtures of these solvents. Non-aqueous solvents can be added to the liquid agents for treating textiles in amounts preferably between 0.5 and 15 wt. %, preferably, however below 12 wt. % and particularly below 9 wt. %.

The viscosity of the agents for treating textiles in the form of liquid detergents or rinse aids can be measured using standard methods (for example using a Brookfield-Viscosimeter LVT-II at 20 rpm and 20° C., spindle 3) and lies preferably in the range from 500 to 5000 mPas for liquid detergents. Preferred agents for treating textiles in the form of liquid detergents have viscosities from 700 to 4000 mPas, particularly preferably from 1000 to 3000 mPas. Preferred agents for treating textiles in the form of rinse aids have viscosities from 20 to 4000 mPas, particularly preferably from 40 to 2000 mPas. The viscosity of rinse aids is particularly preferably from 40 to 1000 mPas.

The addition of pH adjustors can be considered for bringing the pH of the agents for treating textiles into the desired range. Any known acid or alkali can be added, in so far as their addition is not forbidden on technological or ecological grounds or grounds of protection of the consumer. The amount of these adjustors does not normally exceed 7 wt. % of the total formulation.

The pH of the liquid agent for treating textiles in the form of a liquid detergent is preferably between 4 and 10 and particularly preferably between 5.5 and 8.5. The pH of the liquid agent for treating textiles in the form of a rinse aid is preferably between 1 and 6 and particularly preferably between 1.5 and 3.5.

In order to enhance the esthetic impression of the agents for treating textiles, they may be colored with appropriate colorants. Preferred colorants, which are not difficult for the expert to choose, have high storage stability, are not affected by the other ingredients of the agent for treating textiles or by light and do not have any pronounced substantivity for textile fibers, so as not to color them.

Soaps, paraffins or silicone oils, optionally deposited on carrier materials, are examples of the foam inhibitors that can be added to the agents for treating textiles.

Suitable anti-redeposition agents, also referred to as soil repellents, are for example non-ionic cellulose ethers such as methyl cellulose and methyl hydroxypropyl cellulose with a content of methoxy groups of 15 to 30 wt. % and hydroxypropyl groups of 1 to 15 wt. %, each based on the non-ionic cellulose ether, as well as polymers of phthalic acid and/or terephthalic acid or their derivatives known from the prior art, particularly polymers of ethylene terephthalates and/or polyethylene and/or polypropylene glycol terephthalates or anionically and/or non-ionically modified derivatives thereof. Suitable derivatives include the sulfonated derivatives of the phthalic acid polymers and the terephthalic acid polymers.

Optical brighteners can be added to the agents for treating textiles in order to eliminate graying and yellowing of the treated fabric surfaces. These materials absorb onto the fiber and effect a brightening and pseudo bleach effect in that the invisible ultraviolet radiation is converted into visible radiation, wherein the ultraviolet light absorbed from sunlight is irradiated away as weak blue fluorescence and results in pure white for the yellow shade of the grayed or yellowed washing. Suitable compounds derive for example from the substance classes of 4,4′-diamino-2,2′-stilbenedisulfonic acids (flavonic acids), 4,4′-distyrylbiphenylene, methylumbelliferone, coumarone, dihydroquinolinones, 1,3-diarylpyrazolines, naphthoic acid imides, benzoxazole-, benzisoxazole- and benzimidazole-systems as well as heterocyclic substituted pyrene derivatives. The optical brighteners are usually added in amounts between 0% and 0.3 wt. %, based on the finished detergent and cleaning agent.

Graying inhibitors have the function of maintaining the dirt that was removed from the fibers suspended in the washing liquor, thereby preventing the dirt from resettling. Water-soluble colloids of mostly organic nature are suitable for this, for example glue, gelatines, salts of ether sulfonic acids of starches or celluloses, or salts of acidic sulfuric acid esters of celluloses or starches. Water-soluble, acid group-containing polyamides are also suitable for this purpose. In addition, soluble starch preparations and others can be used as the abovementioned starch products, e.g. degraded starches, aldehyde starches etc. Polyvinyl pyrrolidone can also be used. Preference, however, is given to the use of cellulose ethers such as carboxymethyl cellulose (Na salt), methyl cellulose, hydroxyalkyl cellulose and mixed ethers such as methyl hydroxyethyl cellulose, methyl hydroxypropyl cellulose, methyl carboxymethyl cellulose and mixtures thereof, which can be added, for example in amounts of 0.1 to 5 wt. %, based on the agent for treating textiles.

As textile fabrics, particularly of rayon, spun rayon, cotton and their mixtures tend to crease because the individual fibers are sensitive to flection, bending, pressing and squeezing at right angles to the fiber direction, the laundry detergents or cleaning agents can comprise synthetic anti-crease agents. They include for example synthetic products based on fatty acids, fatty acid esters, fatty acid amides, fatty acid alkylol esters, fatty acid alkylol amides or fatty alcohols that have been mainly treated with ethylene oxide, or products based on lecithin or modified phosphoric acid esters.

The agents for treating textiles may contain antimicrobials to control microorganisms. Depending on the antimicrobial spectrum and the action mechanism, antimicrobial agents are classified as bacteriostatic agents and bactericides, fungistatic agents and fungicides, etc. Important representatives of these groups are, for example, benzalkonium chlorides, alkylaryl sulfonates, halophenols and phenol mercuric acetate, wherein these compounds can also be totally dispensed with in the inventive laundry detergents and cleaning agents.

The inventive agents for treating textiles can comprise preservatives, wherein preferably only those are used, which have no or only a slight skin sensitising potential. Examples are sorbic acid and its salts, benzoic acid and its salts, salicylic acid and its salts, phenoxyethanol, 3-iodo-2-propynylbutyl carbamate, sodium N-(hydroxymethyl)glycinate, biphenyl-2-ol as well as mixtures thereof. A suitable preservative is illustrated by the solvent-free, aqueous combination of diazolidinyl urea, sodium benzoate and potassium sorbates (obtainable as Euxyl® K 500 ex Schuelke & Mayr), which can be employed in a pH range up to 7. In particular, preservatives based on organic acids and/or their salts are suitable for preserving the inventive skin-friendly agents for treating textiles.

The detergents and cleaning agents can comprise antioxidants in order to prevent undesirable changes caused by oxygen and other oxidative processes to the agents for treating textiles and/or the treated textile fabrics. This class of compounds includes, for example, substituted phenols, hydroquinones, pyrocatechols and aromatic amines as well as organic sulfides, polysulfides, dithiocarbamates, phosphites, phosphonates and vitamin E.

An increased wear comfort can result from the additional use of antistats that can be included in the laundry detergents and cleaning agents. Antistats increase the surface conductivity and thereby allow an improved discharge of built-up charges. Generally, external antistats are substances with at least one hydrophilic molecule ligand and provide a more or less hygroscopic film on the surfaces. These mainly interface active antistats can be subdivided into nitrogen-containing (amines, amides, quaternary ammonium compounds), phosphorus-containing (phosphoric acid esters) and sulfur-containing (alkyl sulfonates, alkyl sulfates) antistats. Lauryl (or stearyl) dimethyl benzyl ammonium chlorides are suitable antistats for textile fabrics or as additives to agents for treating textiles, resulting in an additional finishing effect.

Silicone derivatives, for example, can be incorporated in the agents for treating textiles to improve the re-wettability of the treated textile fabrics and to facilitate ironing of the treated textile fabrics. By their foam-inhibiting properties, they additionally improve the final rinse behavior of the laundry detergents or cleaning agents. Exemplary preferred silicone derivatives are polydialkylsiloxanes or alkylarylsiloxanes, in which the alkyl groups possess one to five carbon atoms and are totally or partially fluorinated. Preferred silicones are polydimethylsiloxanes that can be optionally derivatized and then are aminofunctional or quaternized or possess Si—OH, Si—H and/or Si—Cl bonds. The viscosities of the preferred silicones at 25° C. are in the range between 100 and 100 000 mPas, wherein the silicones can be added in amounts between 0.2 and 5 wt. % based on the total laundry detergent and cleaning agent.

Finally, the agents for treating textiles can also comprise UV absorbers, which are absorbed on the treated textile fabrics and improve the light stability of the fibers. Compounds, which possess these desired properties, are for example, the efficient radiationless deactivating compounds and derivatives of benzophenone having substituents in position(s) 2- and/or 4. Also suitable are substituted benzotriazoles, acrylates, which are phenyl-substituted in position 3 (cinnamic acid derivatives), optionally with cyano groups in position 2, salicylates, organic Ni complexes, as well as natural substances such as umbelliferone and the endogenous urocanic acid.

Substances can be added to complex heavy metals in order to prevent heavy metal catalyzed decomposition of certain detergent ingredients. Suitable heavy metal sequestrants are, for example, the alkali salts of ethylene diamine tetra acetic acid (EDTA) or of nitrilotriacetic acid (NTA) as well as alkali metal salts of anionic polyelectrolytes such as polyacrylates, polymaleates and polysulfonates.

A preferred class of sequestrants are the phosphonates that are comprised in preferred agents for treating textiles in amounts of 0.01 to 2.5 wt. %, preferably 0.02 to 2 wt. % and particularly 0.03 to 1.5 wt. %. These preferred compounds particularly include organophosphonates such as for example 1-hydroxyethane-1,1-diphosphonic acid (HEDP), aminotri(methylenephosphonic acid) (ATMP), diethylenetriamine penta(methylenephosphonic acid) (DTPMP or DETPMP) as well as 2-phosphonobutane-1,2,4-tricarboxylic acid (PBS-AM), which are mainly added in the form of their ammonium or alkali metal salts.

Solid agents for treating textiles can in addition also comprise neutral filler salts such as sodium sulfate.

The agents for treating textiles according to the invention can be particularly used for cleaning and conditioning textile fabrics.

The rinse aid can be manufactured as an agent for treating textiles according to usual procedures known to the person skilled in the art for the manufacture of rinse aids. For example, this can be carried out by mixing the raw materials, optionally by using high shear mixers. It is recommended to melt the softening component(s) and to subsequently disperse the melt in a solvent, preferably water. The additional ingredients can be incorporated into the rinse aid by a simple blending. The liquid detergent as the agent for treating textiles is manufactured by usual and known methods and processes by, for example by simply blending the ingredients in stirred tanks, wherein water, non-aqueous solvents and surfactants are advantageously present and the other ingredients, including the OH group-containing esterquat, are added portion wise. Separate heating is not required during the preparation, but if desired then the temperature should not exceed 80° C.

The inventive agents for treating textiles are preferably rinse aids and laundry detergents. The rinse aids particularly concern rinse aids that are employed for treating textiles during or after the wash. Laundry detergents can be used for manual or machine washing, particularly of textiles. The laundry detergents or cleaning agents can be for the industrial sector or for the domestic sector. Cleaning agents can also be used for example for cleaning hard surfaces. These can be for example cleaning agents for tableware, which are employed for manual or automatic dishwashing. It can also concern usual industrial or domestic cleaners, with which hard surfaces such as furniture surfaces, floor tiles, tiles, wall and floor coverings can be cleaned. Besides tableware, hard surfaces also refer to all usual hard surfaces, in particular of glass, ceramic, plastic or metal, in the household and in industry. The agents for treating textiles or surfaces can be solid or liquid formulations, wherein solid formulations can be present as a powder, granulate, extrudate, as a tablet or as a compressed and/or molten molded object. The liquid formulations can be solutions, emulsions, dispersed suspensions, micro-emulsions, gels or pastes.

Accordingly, as the agent for treating surfaces, the agent can comprise usual ingredients of cleaning agents in usual quantities.

For example, as cleaning agents, the agents for treating surfaces can comprise alkyl ether sulfates, alkyl sulfonates and/or aryl sulfonates, alkyl sulfates, amphoteric surfactants, anionic surfactants, non-ionic surfactants, cationic surfactants, solvents, thickeners, dicarboxylic acid (salts) and further auxiliaries and additives. These additional ingredients have in part already been mentioned in detail and are likewise valid for use in cleaning agents (see e.g. non-ionic surfactants). UV stabilizers, perfume, opacifying agents (INCI); (for example glycol distearate, e.g. Cutina® AGS from Henkel KGaA, or mixtures comprising them, e.g. Euperlane® from Henkel KgaA), SRP (soil repellent polymers), PEG terephthalates, colorants, bleaching agents (e.g. hydrogen peroxide), corrosion inhibitors, preservatives (e.g. industrial 2-bromo-2-nitropropane-1,3-diol also known as Bronopol (CAS 52-51-7) that is commercially available as Myacide® BT or as Boots Bronopol BT from Boots) as well as skin improvers or skin care additives (e.g. dermatologically active substances such as vitamin A, vitamin B2, vitamin B12, vitamin C, vitamin E, D-panthenol, sericerin, collagen partial hydrolysate, various vegetal protein partial hydrolysates, protein hydrolysate-fatty acid condensates, liposomes, polypropylene glycol, Nutrilan™, Chitosan™, cholesterin, vegetal and animal oils such as e.g. lecithin, soya oil, etc., vegetal extracts such as e.g. aloe vera, azulene, hamamelis extracts, algae extracts, etc., allantoin, A.H.A complexes) in particular can be comprised as auxiliaries or additives, particularly in manual dishwasher detergents and cleaning agents for hard surfaces, usually in amounts not exceeding 5 wt.-%. Minor amounts of enzymes can be added to increase the performance. Proteases (e.g. BLAP (Henkel), Savinase (NOVO), Durazym (NOVO), Maxapemm, etc.), Amylases (e.g. Fermamyl (NOVO), etc.), Lipases (e.g. Lipolase (NOVO), etc.), Peroxidases, Gluconases, Cellulases, Mannases, etc., are preferred in quantities of preferably 0.001 to 1.5% and particularly preferably less than 0.5%.

The invention further relates to the use of the agent for treating textiles or surfaces for washing textiles or cleaning hard surfaces. The invention further relates to the use of the agent for an improved fragrance yield on textiles or for an improved delayed fragrance on textiles or on hard surfaces.

In addition, the invention relates to a method for prolonging the perception of fragrance of laundry detergents or cleaning agents, rinse aids or solid surfaces treated with these agents.

Moreover the invention also relates to a method for manufacturing an agent for treating textiles or surfaces, in which at least one fragrance and at least one OH group-containing esterquat corresponding to Formula (I) are mixed together.

Other than where otherwise indicated, or where required to distinguish over the prior art, all numbers expressing quantities of ingredients herein are to be understood as modified in all instances by the term “about”. As used herein, the words “may” and “may be” are to be interpreted in an open-ended, non-restrictive manner. At minimum, “may” and “may be” are to be interpreted as definitively including, but not limited to, the composition, structure, or act recited.

As used herein, and in particular as used herein to define the elements of the claims that follow, the articles “a” and “an” are synonymous and used interchangeably with “at least one” or “one or more,” disclosing or encompassing both the singular and the plural, unless specifically defined herein otherwise. The conjunction “or” is used herein in both in the conjunctive and disjunctive sense, such that phrases or terms conjoined by “or” disclose or encompass each phrase or term alone as well as any combination so conjoined, unless specifically defined herein otherwise.

The description of a group or class of materials as suitable or preferred for a given purpose in connection with the invention implies that mixtures of any two or more of the members of the group or class are equally suitable or preferred. Description of constituents in chemical terms refers unless otherwise indicated, to the constituents at the time of addition to any combination specified in the description, and does not necessarily preclude chemical interactions among the constituents of a mixture once mixed. Steps in any method disclosed or claimed need not be performed in the order recited, except as otherwise specifically disclosed or claimed.

Changes in form and substitution of equivalents are contemplated as circumstances may suggest or render expedient. Although specific terms have been employed herein, such terms are intended in a descriptive sense and not for purposes of limitation. The invention is illustrated in more detail by means of the following examples.

Synthesis of OH group-containing Esterquats

### a) Preparation of the Ester Amine

Hypophosphoric acid (50% conc., 1 ml) was added to fatty acid (0.5 mol) and diethanol/methylamine (DEMA, 0.25 mol) under a nitrogen atmosphere in a four necked flask equipped with a pivot stirrer shaft, water separator and Dimroth condenser. Toluene (25 ml) was then added. For fatty acids that are solid at room temperature, the flask was slowly heated beforehand until the reaction mixture liquefies. Once no more water separates off, the temperature was increased to 150° C. until the water separation also finishes at this temperature. The reaction was then interrupted and the solvent removed under vacuum. The products that solidified on cooling were transferred into a wide beaker. On cooling, the product can be uniformly dispersed on the glass wall by rotating the beaker. This is advantageous for the complete removal of the solvent in the vacuum dryer.

b) Quaternization of the Ester Amine to the Esterquat

The ester amine obtained from a) was placed in a four necked flask equipped with a pivot shaft stirrer, Dimroth condenser, nitrogen supply tube, temperature sensor and heating mantle and optionally heated until melted. Dimethyl sulfate was then added causing the reaction temperature to increase. Once the temperature fell again, stirring was continued for 1 hour at 80° C. The resulting product was used without further purification.

Analytical Determination of the Yield of Perfume Oil from Detergents

### b) Perfume Oil Mixture

A perfume oil mixture was prepared from hexyl acetate, Herbavert, dihydromyrcenol, tetrahydromyrcenol, cyclovertal, linalool, phenylethyl alcohol, citronellol, citral, geraniol, hydroxycitronellal, isobornyl acetate, OTBCA (2-tert-butylcyclohexyl acetate), PTBCA (4-tert-butylcyclohexyl acetate), aldehyde C12 MNA (2-methylundecanal), alpha-ionone, Acedyl, Floramat, Acetate PA (allylphenoxy acetate), lilial, norlimbanol, Hedione, benzophenone, alpha-amylcinnamaldehyde, lyral, Iso E Super (7-acetyl-1,2,3,4,5,6,7,8-octahydro-1,1,6,7-tetramethylnaphthalene), cyclohexyl salicylate, Habanolide, benzyl salicylate, ethylene brassilate.

All perfume oils were present in identical weight fractions and were analytically detectable.

b) Textile Used:

Woven towel (baled goods) was used for the textile finishing. 1.8 kg of the material was pre-washed with 30 g of a commercially available heavy duty laundry detergent in a washing machine (Miele, type Novotronic W 363) using the boil-coloreds program at 95° C. Rinsing was carried out five times. The toweling material was hung on the line to dry at 20° C. and 65% relative humidity.

c) Textile Finishing:

Samples (10×10 cm, 4.8 g textile) were cut from the pre-washed toweling material. Three test specimens (moist and dry) were prepared per product. The wash liquor ratio material:treatment solution was 1:5, wherein the wash liquor was added based on the calculation of 36 mL rinse aid [15% Esterquat, 0.9% abovementioned perfume oil mixture and 0.2% MgCl₂*6H₂O]/5 L water. The prepared test specimens were placed individually in flat glass dishes into which 24 g treatment wash liquor had been poured, and then the treated toweling test specimens were spun for one minute in a domestic spin-dryer (1400 RPM). The test specimens, which should be examined when moist, were individually packed in glass bottles immediately after spinning. The remainder was laid flat onto metal grids, stored in a conditioning chamber at 20° C. and 65% relative humidity for 24 hours and then individually packaged in glass bottles.

d) Analysis:

The moist cloths were immediately extracted with pentane by means of an ASE extraction (ASE=accelerated solvent extraction). The solution was then concentrated down at 30° C. to 2 ml. The samples were then examined on a 30 m DB5MA column (film thickness 1 μm) by means of GC-MS coupling in EI mode (electron ionisation) and parallel switched FID (flame ionisation detection).

The cloths were then extracted by SPE (solid phase extraction) according to the SBSE (stir bar sorptive extraction) method by means of a Twister™ from the Gerstel Company. For this the dry cloths were extracted after activation (4 h at 80° C.) in the steam room (enrichment of the twister: 24 h at room temperature). In the solid phase extraction, the concentration of the analytes in a liquid or in the gas phase is continually reduced by adsorption on a solid adsorbent, such that even odoriferous substances with a very low vapor pressure or very low solubility can continually diffuse out of the textile fabrics into the liquid or gas phase. Here, the analytical equipment is: Thermodesorption apparatus (Gerstel Company) with “TDS 2” auto sampler and cooled injection system, cooled with liquid nitrogen; # gas chromatograph HP 6890 Quadrupol-MSD HP 5973 TDS tube: pyrex glass, 1=178 mm, dinner=4 mm headspace vials: V=22 cm³, with aluminum coated Teflon septum.

After extraction, the samples were examined on a 30 m DB5MS column (film thickness 1 μm) by means of GC-MS coupling in EI mode (electron ionisation) and parallel switched FID (flame ionisation detection).

The analytical results are presented in Table 1.

As shown in Table 1, fragrances are adsorbed better on textiles that were treated with agents that possess OH group-containing esterquats. Table 1 also shows that the perfume oil yield on the moist textile is significantly increased by the addition of the OH group-containing esterquats corresponding to Formula (I). The perfume oil content subsequently remaining on the washing is correspondingly higher than for esterquats that do not carry OH groups.

TABLE 1

| | Concentration of Perfume oil on Textile [μg/g] | | | |
|---|---|---|---|---|
| | EQ1 | EQ2 | Rewoquat WE 18 | Stearic acid |
| Spun moist textile | 65.8 | 44.3 | 37.5 | 34.5 |
| Dryed textile after 1 day | 6.0 | 8.9 | 4.1 | 4.39 |

EQ1 = Esterquat of Formula (I) with
R₁ = —CH₃
R₂ = —CH₂—CH₂—OH
R₃ = R₄ = —(CH₂)ₘ—A—C(O)—Z, with A = —O—, m = 2 and
Z = saturated C₁₇-alkyl chain with OH group on the C-11 atom.
EQ2 = Esterquat of Formula (I) with
R₁ = —CH₃
R₂ = —CH₂—CH₂—OH
R₃ = R₄ = —(CH₂)ₘ—A—C(O)—Z, with A = —O—, m = 2 and
Z = unsaturated C₁₇-alkyl chain with OH group on the C-11 atom, wherein the double bond is on the C-8 atom.

3. Olfactory Investigations

Washing conditions

| | |
|---|---|
| Washing machine: | Miele Novotronic W 308 |
| Primary wash performance: | Soaking step Normal program boil-coloreds |
| | wash |
| Wash temperature: | 40° C. |
| Volume of wash liquor: | 15 l |
| Water hardness: | 15°dH |
| Amount of laundry: | 3.5 kg clean laundry |
| Fabric: | toweling-soap hand towel |

c) Procedure:

The textiles were washed with a laundry detergent, then rinsed with 36 ml rinse aid (composition of the rinse aid: 13% esterquat, 0.9% perfume oil, 0.2% MgCl₂*6H₂O) and dried. A parallel test was carried out with the classic rinse aids and a rinse aid that was manufactured from the esterquat according to the invention. New cloths were produced for the initial and 1 week values for each subject. After drying over night, the finished cloths were smelt the next morning (AW). In order to assess a long lasting effect, the cloths were also smelt after 7 days. The textiles were presented for comparison to a panel of experts composed of 30 people. The evaluation was made on a scale of 1 to 5, wherein 5 means “very intensive smell” and 1 means “hardly any smell or odourless”.

1 no fragrance, hardly noticeable

2 little fragrance noticeable

3 somewhat to smell

4 good scent

5 very good scent

TABLE 2

| Intensity: | | | | |
|---|---|---|---|---|
| orchids &lotus | | Moist | Dry washing | Dry washing |
| blossom | Product | washing | (1 d) | (7 d) |
| Rewoquat WE 18 | 4.7 | 4.7 | 3.9 | 3.6 |
| EQ1 | 4.3 | 4.7 | 5 | 4.1 |
| EQ2 | 4.3 | 4.6 | 4.2 | 4.3 |

Rewoquat WE 18 is comparable with EQ1 and EQ2 in the product and on the moist washing (Table 2). However, Rewoquat WE18 is clearly inferior to and EQ2 after drying. The comparison between Rewoquat WE18 and EQ1 and EQ2 clearly shows here that an OH group on the side chain of the ester of the esterquat affords an improved fragrance delay contrary to esterquats without OH groups.

What is claimed:

1. An agent for treating textiles or surfaces, comprising at least one fragrance and at least one OH group-containing esterquat of the general Formula (I):
[N⁺R₁R₂R₃R₄]X⁻ (I)
in which
R₁ stands for an alkyl group containing 1 to 4 carbon atoms or a hydroxyalkyl group containing 1 to 4 carbon atoms,
R₂, R₃, R₄ independently of each other stand for an alkyl group containing 1 to 4 carbon atoms, a hydroxyalkyl group containing 1 to 4 carbon atoms or —(CH₂)ₘ-A-C(O)-Z or —(CH₂)ₘ—C(O)-A-Z, wherein A stands for —O, —S—, —NR₅— with R₅=H or an alkyl group containing 1 to 4 carbon atoms, Z stands for a saturated or unsaturated alkyl group containing 8 to 22 carbon atoms that comprises at least one OH group in the side chain, and m stands for a whole number in the range 1 to 3, and
X stands for an anion of an inorganic or organic acid,
wherein at least one group R₂, R₃ or R₄ stands for —(CH₂)ₘ-A-C(O)-Z or —(CH₂)ₘ—C(O)-A-Z.

2. The agent of claim 1, wherein the OH group-containing esterquat has a degree of OH substitution of 1 to 3.

3. The agent of claim 2, wherein the degree of OH substitution is 1.7 to 2.2.

4. The agent of claim 1, wherein Z comprises an unbranched, saturated or unsaturated C₈—, C₉—, C₁₀—, C₁₁—, C₁₂—, C₁₃—, C₁₄—, C₁₅—, C₁₆—, C-₁₇—, C₁₈—, C₁₉—, C_{2O}—, C₂₁—, or C₂₂— group.

5. The agent of claim 1, wherein X comprises a halide, sulfate, methosulfate, phosphate, formate, propionate or acetate.

6. The agent of claim 1, comprising 0.5% to 40% by weight of the at least one OH group-containing esterquat.

7. The agent of claim 6, comprising 2.5% to 30% by weight of the at least one OH group-containing esterquat.

8. The agent of claim 7, comprising 3.5% to 20% by weight of the at least one OH group-containing esterquat.

9. The agent of claim 1, comprising 0.01% to 5% by weight of the at least one fragrance.

10. The agent of claim 9, comprising 0.1% to 3% by weight of the at least one fragrance.

11. The agent of claim 10, comprising 0.5% to 2% by weight of the at least one fragrance.

12. The agent of claim 1, wherein the fragrance comprises a synthetic or natural ester, ether, aldehyde, ketone, alcohol, hydrocarbon, acid, carboxylic acid ester, aromatic hydrocarbon, aliphatic hydrocarbon, saturated hydrocarbon, or unsaturated hydrocarbon.

13. The agent of claim 1, comprising a laundry detergent, rinse aid, softening laundry detergent, or laundry detergent auxiliary.

14. The agent of claim 1, further comprising an anionic, cationic, non-ionic, zwitterionic, or amphoteric surfactant.

15. The agent of claim 14, wherein the surfactant comprises a non-ionic or a cationic surfactant.

16. The agent of claim 1, having a solid form.

17. The agent of claim 1, having a powder, granulate, extrudate, tab, tablet, compressed, or molded form.

18. The agent of claim 1, having a liquid form.

19. The agent of claim 1, having a solution, emulsion, dispersion, suspension, micro-emulsion, gel or paste form.

20. A method of improving fragrance yields or fragrance delays on textiles, comprising contacting a textile with an effective amount of an agent for treating textiles, said agent comprising at least one fragrance and at least one OH group-containing esterquat of the general Formula (I):
[N⁺R₁R₂R₃R₄]X⁻ (I)
in which
R₁ stands for an alkyl group containing 1 to 4 carbon atoms or a hydroxyalkyl group containing 1 to 4 carbon atoms,
R₂, R₃, R₄ independently of each other stand for an alkyl group containing 1 to 4 carbon atoms, a hydroxyalkyl group containing 1 to 4 carbon atoms or —(CH₂)ₘ-A-C(O)-Z or —(CH₂)ₘ—C(O)-A-Z, wherein A stands for —O, —S—, —NR₅— with R₅=H or an alkyl group containing 1 to 4 carbon atoms, Z stands for a saturated or unsaturated alkyl group containing 8 to 22 carbon atoms that comprises at least one OH group in the side chain, and m stands for a whole number in the range 1 to 3, and
X stands for an anion of an inorganic or organic acid,
wherein at least one group R₂, R₃ or R₄ stands for —(CH₂)ₘ-A-C(O)-Z or —(CH₂)ₘ—C(O)-A-Z.
